(19) United States
(12) Patent Application Publication
(10) Pub. No.: US 2025/0028180 A1
(43) Pub. Date: Jan. 23, 2025

(54) BEAM DELIVERY SYSTEM FOR PROBE WITH MULTI-CORE FIBER

(71) Applicant: Alcon Inc., Fribourg (CH)

(72) Inventors: Alireza Mirsepassi, Irvine, CA (US); Mark Harrison Farley, Laguna Hills, CA (US); Ronald T. Smith, Irvine, CA (US)

(21) Appl. No.: 18/760,157

(22) Filed: Jul. 1, 2024

Related U.S. Application Data
(60) Provisional application No. 63/514,616, filed on Jul. 20, 2023.

(51) Int. Cl. G02B 27/09

(52) U.S. Cl. CPC G02B 27/0916; G02B 27/0944; G02B 27/0961

(57) ABSTRACT

A beam delivery system for a probe includes a plurality of laser sources configured to generate a respective incident beam, including a first laser source generating a first incident beam and a second laser source generating a second incident beam. The system includes routing structures respectively positioned along a path of the respective incident beam. An optical subsystem is adapted to sequentially direct a respective output beam from the routing structures into each core of a multi-core fiber in communication with the probe. The first routing structure and the second routing structure respectively include an array of optical elements adapted to be synchronously moved such that the first incident beam and the second incident beam encounter an identical member of the array at a same time.

### INTRODUCTION

The present disclosure relates generally to an optical surgical probe. More specifically, the disclosure relates to a beam delivery system for a probe with a multi-core fiber. Optical probes may be used for various purposes during ophthalmic surgery. For example, during laser photocoagulation, a laser probe may be employed to cauterize blood vessels at burn spots across a retina of an eye. Some types of laser probes may burn multiple spots at a time, such a multi-spot laser probes, resulting in faster and more efficient photocoagulation. A multi-spot laser probe may split a single laser beam into multiple laser beams and deliver the beams simultaneously to an array of optical fibers that yield a spot pattern at a target location. Delivery of the laser beam to the optical fibers may be challenging for various reasons.

### SUMMARY

Disclosed herein is a beam delivery system for a probe. The system includes a plurality of laser sources configured to generate a respective incident beam, including a first laser source generating a first incident beam and a second laser source generating a second incident beam. The system includes routing structures respectively positioned along a path of the respective incident beam, including a first routing structure in the path of the first incident beam and a second routing structure in the path of the second incident beam. An optical subsystem is adapted to direct a respective output beam from the routing structures into a multi-core fiber in communication with the probe. The respective output beam is sequentially directed into each core of the multi-core fiber. The first and the second routing structures respectively include an array of optical elements. The first and the second routing structures are adapted to be synchronously moved such that the first incident beam and the second incident beam encounter an identical member of the array at the same time.

The optical elements include at least one of a refractive wedge, a diffractive element, and a reflective element. In some embodiments, each of the routing structures are integrally formed such that the optical elements are rigidly attached to one another. The optical elements in the array may be arranged within a plane substantially perpendicular to the respective incident beam, with the routing structures being sequentially translated across each of the optical elements. Each of the optical elements may be configured to have a diffractive structure or a refractive structure that is fixed across a spatial area of the optical elements.

In another embodiment, the routing structures respectively include a rotating disk defining a planar input surface perpendicular to the respective incident beam, with the optical elements being arranged in a radial configuration. Here, the routing structures are sequentially rotated across each of the optical elements along a rotational axis perpendicular to the plane of the rotating disk. The optical elements may include substantially conical wedge elements respectively defining an output surface. The optical elements may be configured to have a refractive structure that varies along a circumferential direction relative to the rotational axis, the refractive structure being fixed along a radial direction relative to the rotational axis. A respective corrective lens may be positioned along the path of the respective incident beam, prior to the respective incident beam encountering the routing structures.

In some embodiments, the optical elements include oscillating scanning mirrors synchronously movable between respective positions corresponding to each core of the multi-core fiber. The system may include a controller configured to pulse the plurality of laser sources such that each laser beam generated by the plurality of laser sources is only active when the laser beam is residing within a respective core region of the multi-core fiber. The controller has at least one processor and at least one non-transitory, tangible memory on which instructions are recorded. In some embodiments, the time-averaged power of the laser beam equals a continuous wave power of a non-pulsed beam.

Disclosed herein is a beam delivery system for a probe. The system includes a plurality of laser sources configured to generate a respective incident beam, including a first laser source generating a first incident beam and a second laser source generating a second incident beam. Routing structures are respectively positioned along a path of the respective incident beam, including a first routing structure in the path of the first incident beam and a second routing structure in the path of the second incident beam, the probe being in communication with a multi-core fiber. The system includes an optical subsystem adapted to sequentially direct a respective output beam from the routing structures respectively into each core of the multi-core fiber. The first routing structure and the second routing structure respectively include an array of optical elements synchronously movable between respective positions corresponding to each core of the multi-core fiber. In one embodiment, the optical elements are oscillating scanning mirrors.

The above features and advantages and other features and advantages of the present disclosure are readily apparent from the following detailed description of embodiments for carrying out the disclosure when taken in connection with the accompanying drawings.

### BRIEF DESCRIPTION OF THE DRAWINGS

FIG. 1A is a schematic fragmentary diagram of a beam delivery system with routing structures, in accordance with a first embodiment;

FIG. 1B illustrates the beam delivery system of FIG. 1A, with the routing structures translated to a different position;

FIG. 2A is a schematic side view illustrating an example routing structure employable in the system of FIGS. 1A, 1B;

FIG. 2B is a schematic perspective bottom view of the routing structure of FIG. 2A;

FIG. 3A is a schematic fragmentary diagram of a second embodiment of a beam delivery system having routing structures;

FIG. 3B illustrates the beam delivery system of FIG. 3A, with the routing structures rotated to a different position;

FIG. 4A is a schematic fragmentary perspective top view of an example routing structure employable in the system of FIGS. 3A, 3B;

FIG. 4B is a schematic perspective bottom view of the routing structure of FIG. 4A;

FIG. 5A is a schematic fragmentary diagram of a third embodiment of a beam delivery system having routing structures; and

FIG. 5B illustrates the beam delivery system of FIG. 5A, with the routing structures in a different position.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

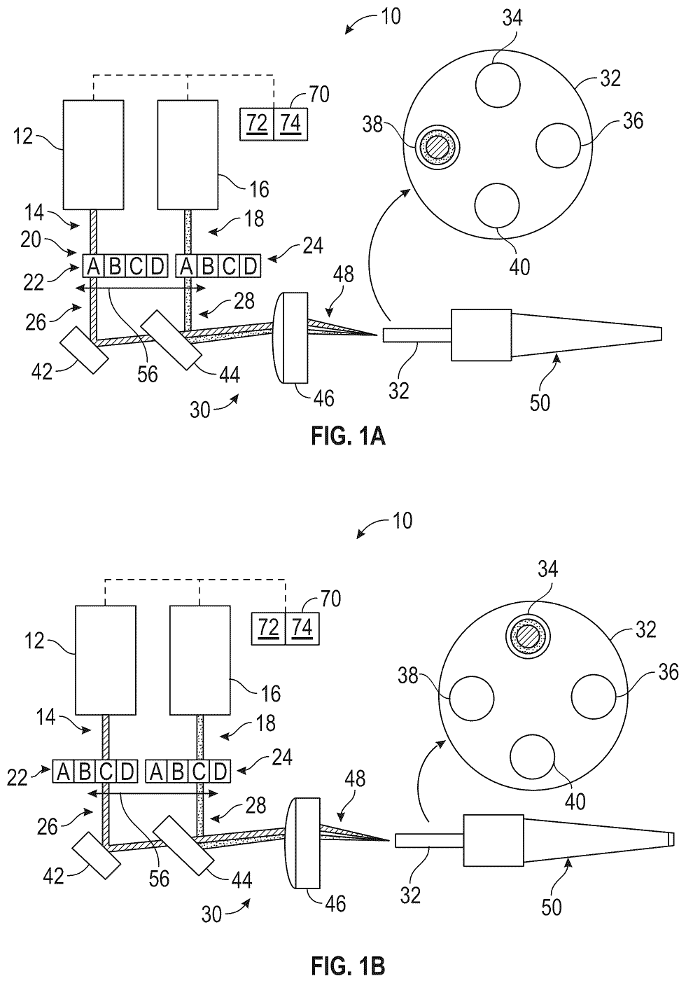

Referring to the drawings, wherein like reference numbers refer to like components, FIGS. 1A-1B schematically illustrate a beam delivery system 10 with a plurality of laser sources configured to generate a respective incident beam. In the embodiment shown, the beam delivery system 10 includes a first laser source 12 generating a first incident beam 14 and a second laser source 16 generating a second incident beam 18. The first laser source 12 and the second laser source 16 may be any suitable light source that can generate a laser beam. The first laser source 12 and the second laser source 16 may have a laser shutter that can switch the beam on and off. The number of laser sources may be varied based on the application at hand. It is understood that the FIGS. are not drawn to scale.

The system 10 includes routing structures 20 respectively positioned along a path of the respective incident beam. Referring to FIGS. 1A, 1B, the routing structures 20 include a first routing structure 22 in the path of the first incident beam 14 and a second routing structure 24 in the path of the second incident beam 18. The routing structures 20 may be identical in some embodiments. FIG. 1B illustrates the system 10 with the first and second routing structures 22, 24 having been moved to a different position relative to their position in FIG. 1A. As described below, the system 10 employs multiple light sources, each light source having a moving routing structure acting simultaneously with the routing structures of other light sources, to sequentially direct each incident beam into a multi-core fiber 32 in communication with a laser probe 50. In some embodiments, the multi-core fiber 32 may be integrally formed with the laser probe 50. In some embodiments, the multi-core fiber 32 may be operatively connected to the laser probe 50.

The intermediate beams 26, 28 exiting respectively the first and second routing structures 22, 24 are directed by an optical subsystem 30 into each core of a multi-core fiber 32, for transmission towards a target site. The target site may be an anatomical location on a patient, such as the posterior region of an eye. In the example shown, the multi-core fiber 32 includes a first core 34, a second core 36, a third core 38, and a fourth core 40 (see FIGS. 1A, 1B). Each of the cores in the multi-core fiber 32 may act as a waveguide such that light may independently propagate through those cores. While the examples shown below employ a probe fiber with four fiber cores and a routing structure with four optical elements, it is understood that the number of optical elements and fiber cores may be varied based on the application at hand.

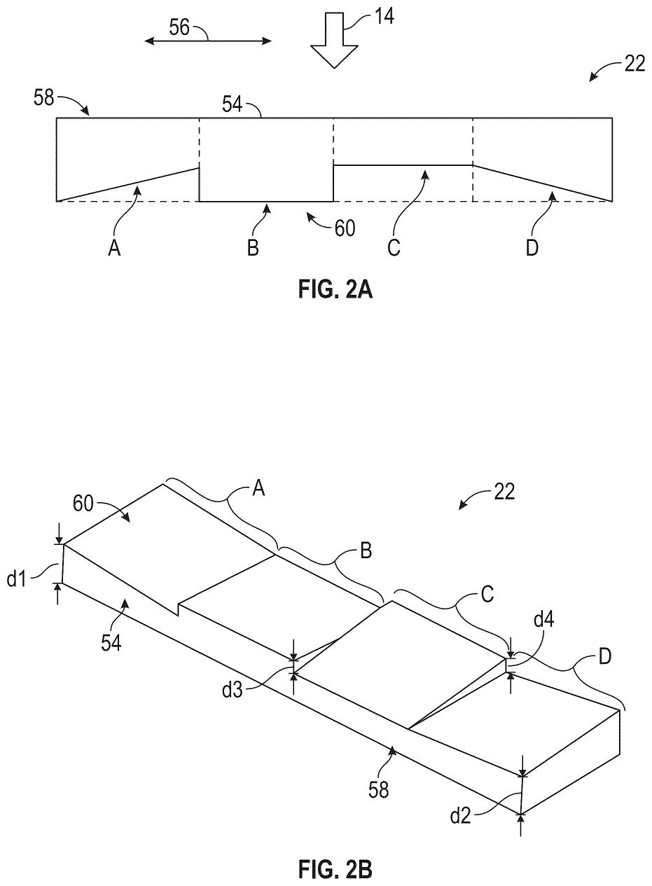

In the example shown in FIGS. 1A, 2A, the optical subsystem 30 includes a relay mirror 42 that directs the first incident beam 14 towards a dichroic mirror 44. The dichroic mirror 44 directs the first incident beam 14 as well as the second incident beam 18 towards a condensing lens 46 that refocuses an output beam 48 onto the multi-core fiber 32.

The system 10 provides a scanned beam delivery system which may be implemented in a surgical laser source with multi-core fiber optic transmission to patient internal or external treatment sites. The multi-core fiber 32 transmits the output beam 48 towards the target site through the multi-spot laser probe 50. The multi-spot laser probe 50 enables the burning of multiple spots at a time, making the procedure faster. The output beam 48 forms a pattern at the target site that matches the laser spot and fiber patterns. The output of the multi-spot laser probe 50 may be used for any suitable purpose, including but not limited to, performing photocoagulation on the retina of the eye.

An example side view of the first routing structure 22 is shown in FIG. 2A. A schematic perspective bottom view of the routing structure 22 is shown in FIG. 2B. The routing structure 22 includes an array of optical elements 54. In the embodiment shown, the optical elements 54 are refractive wedges. In another embodiment, the optical elements 54 may incorporate diffractive elements. Here, the first routing structure 22 incorporates a diffractive structure to diffract beam 14 with a first wavelength. The second routing structure 24 includes a similar diffractive structure, with a different wavelength, to diffract beam 18. The spacings of the diffractive features in the first and second routing structures 22 and 24 would be different from each other (because the respective wavelengths are different) in order to diffract intermediate beams 26, 28 in the same direction. The diffractive elements direct the intermediate beams 26, 28 in the same direction as shown in FIG. 2A. It is understood that the optical elements 54 may incorporate other types of optical structures available to those skilled in the art.

The first and second routing structures 22, 24 are integrally formed such that the optical elements 54 are rigidly attached to one another. Referring to FIGS. 1A, 1B, 2A, 2B, the first and second routing structures 22, 24 each include a first element A, a second element B, a third element C, and a fourth element D. Referring to FIG. 2A, the optical elements 54 arranged along an axis 56 (shown also in FIG. 1A-1B) perpendicular to the incident beam 14. Here, the input surface 58 of the first routing structure 22 is uniform and perpendicular to the first incident beam 14, as shown in FIG. 2A. The output surface 60 of the first routing structure 22 is non-uniform, as shown in FIG. 2B. In some embodiments, the planar surface 58 is the first surface that the beam 14 (or beam 18) encounters. In other embodiments, the planar surface 58 may be the second surface that the beam 14 (or beam 18) encounters and the prismatic surface is the first surface. In some embodiments, the first and second incident beams 14 and 18 may be oriented non-perpendicularly or away from vertical on the screen, such that they are traveling towards the relay mirror 42 and dichroic mirror 44 in a direction that is non-perpendicular to the routing structures 22, 24, respectively.

The first routing structure 22 and the second routing structure 24 are sequentially translated along the axis 56 across each of the optical elements 54. The first and second routing structures 22, 24 are adapted to be synchronously moved such that the first and second incident beams 14, 18 encounter an identical member of the array at the same time. The system 10 may employ time-division-multiplexing, including placing multiple data streams in a single signal by separating the signal into many segments, each having a relatively short duration. Each individual data stream may be reassembled at the receiving end based on the timing.

Referring to FIG. 1A, when the first and second incident beams 14, 18 impinge upon element A of the first and second routing structures 22, 24, respectively, the output beam is directed to the first core 34. In FIG. 1B, the first and second incident beams 14, 18 impinge upon element C of the first and second routing structures 22, 24, respectively, and the output beam being directed to the third core 38. Similarly, when the first and second incident beams 14, 18 impinge upon elements B of the first and second routing structures 22, 24, respectively, the respective output beam is directed to the second core 36. When the first and second incident beams 14, 18 respectively impinge upon element D (of the first and second routing structures 22, 24), the output beam 48 is directed to the fourth core 40. Thus, the first and second incident beams 14, 18 impinge upon elements A, B, C, D in sequence, and the output beam 48 is respectively directed to the first core 34, second core 36, third core 38, and fourth core 40, in sequence.

The optical elements 54 are configured to have a fixed diffractive structure or refractive structure across a spatial area of each of the optical elements 54 such that the respective beams (e.g., intermediate beams 26, 28 exiting the first and second routing structures 22, 24) outputted by each of the optical elements 54 is the same relative to the orientation of the respective incident beam (e.g., first and second incident beam 14, 18). In other words, the difference in direction of the incident beam refracted (or diffracted) from each of the optical elements 54, relative to the incident beam direction, will remain unchanged if the incident beam is fully within the area of the individual optical elements 54. In the embodiment shown, the elements A, B, C, and D are identical but are oriented differently spatially within the array. Thus, distances d1 and d2 are the same in FIG. 2B. Distances d3 and d4 are the same in FIG. 2B. For example, the spatial orientation of element A differs from the spatial orientation of element B by a 90° counterclockwise rotation. Going from the spatial orientations of elements B to C involves a 180° rotation. Going from the spatial orientations of elements C to D involves a 90° clockwise rotation.

As the optical elements 54 are linearly translated and each incident beam transitions between an initial element (e.g., element A) and its adjacent element (e.g., element B), there is a smooth linear transition in beam power between the power to the refracted beam (or diffracted) of the initial element and the refracted (or diffracted) beam of the adjacent element until all of the power is within the refracted (or diffracted) beam of the adjacent element. During this transition process, the positions of each of the two beams do not change, only the power apportionment between them.

The system 10 may include a controller 70 configured to pulse the plurality of laser sources such that each laser beam generated by the plurality of laser sources is only active when the laser beam is residing within a respective core region of the multi-core fiber 32. In other words, the beam is switched on and off to yield a laser spot pattern that matches a fiber pattern of the multi-core fiber 32. The controller 70 has at least one processor 72 and at least one memory 74 (or non-transitory, tangible computer readable storage medium) on which instructions are recorded for a method of pulsing the plurality of laser sources. The memory 74 can store controller-executable instruction groups, and the processor 72 can execute the controller-executable instruction groups stored in the memory 74.

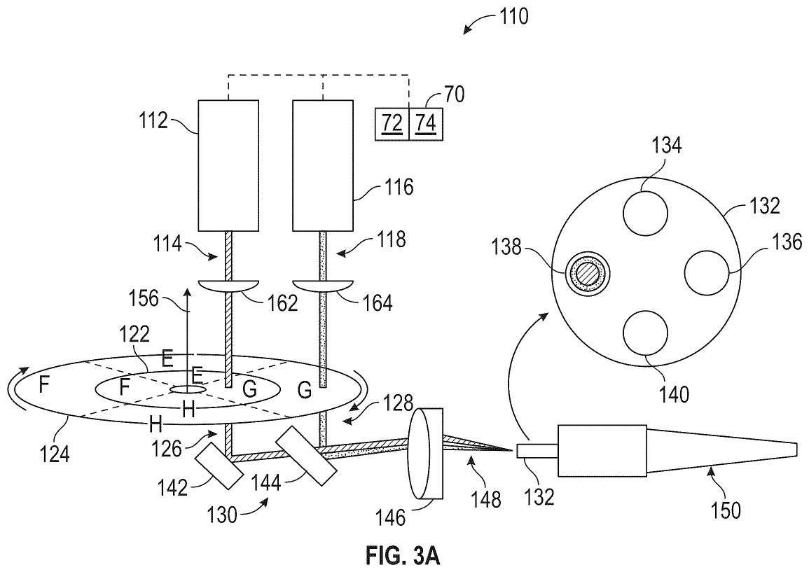

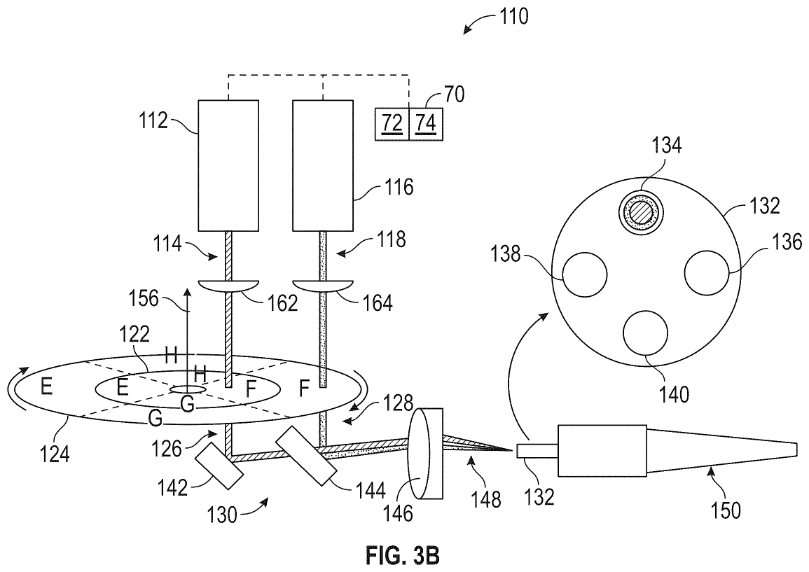

Referring now to FIGS. 3A-3B, a beam delivery system 110 in accordance with a second embodiment is shown. The beam delivery system 110 includes a first laser source 112 generating a first incident beam 114 and a second laser source 116 generating a second incident beam 118. The system 110 includes routing structures 120 respectively positioned along a path of the respective incident beam, including a first routing structure 122 in the path of the first incident beam 114 and a second routing structure 124 in the path of the second incident beam 118. In some embodiments, the routing structures 120 may be identical.

The intermediate beams 126, 128 exiting respectively the first and second routing structures 122, 124 are directed by an optical subsystem 130 into each core of a multi-core fiber 132 in communication with a laser probe 150, for transmission to a target site. The target site may be an anatomical location on a patient, such as the posterior region of an eye. The multi-core fiber 132 may be integrally formed with the laser probe 150. The multi-core fiber 132 may be operatively connected to the laser probe 150. In the example shown, the multi-core fiber 132 includes a first core 134, a second core 136, a third core 138, and a fourth core 140 (see FIGS. 1A, 1B). Each of the cores in the multi-core fiber 132 may act as a waveguide such that light may independently propagate through those cores. As noted above, it is understood that the number of optical elements and fiber cores may be varied based on the application at hand.

In the example shown, the optical subsystem 130 includes a relay mirror 142 that reflects the first incident beam 114 towards a dichroic mirror 144. The dichroic mirror 144 transmits the first incident beam 114 and reflects the second incident beam 118 towards a condensing lens 146 that refocuses an output beam 148 onto the multi-core fiber 132.

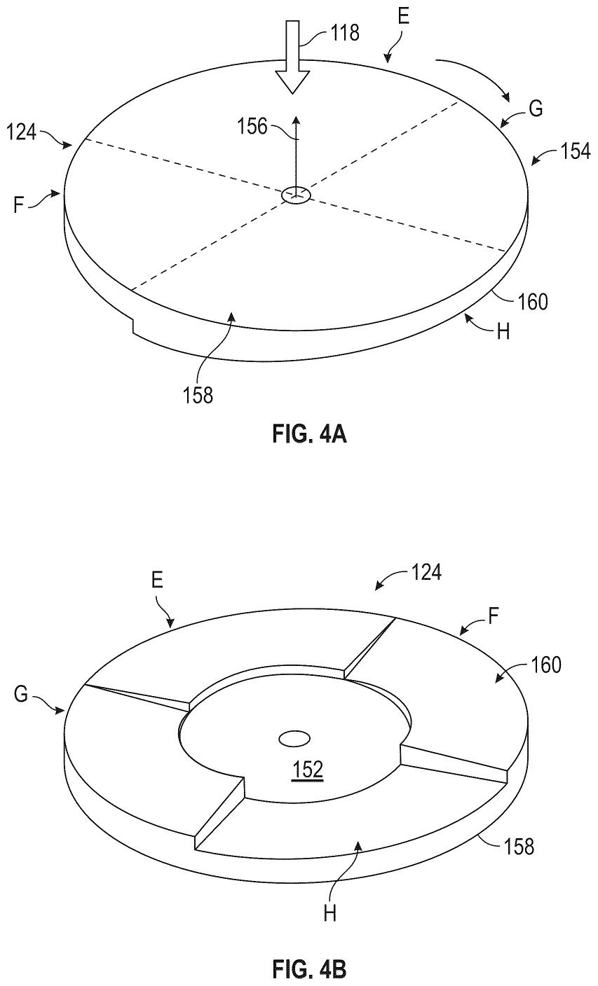

Referring to FIGS. 3A-3B, the first and second routing structures 122, 124 each include a first element E, a second element F, a third element G, and a fourth element H. FIG. 4A show a top view and a bottom view of an example routing structure 124 employable in the system 110. The second routing structure 124 includes an array of optical elements 154 which may include refractive wedge elements, rotary diffractive optical elements, and other optical structures available to those skilled in the art.

Referring to FIGS. 3A, 3B, the first routing structure 122 (and the second routing structure 124) is sequentially rotated across each of the optical elements 154 along a rotational axis 156 that is in a plane perpendicular to the path of the incident beam 114. In the embodiment shown, the first and second routing structures 122, 124 are integrally formed such that the optical elements 154 are rigidly attached to one another and arranged in a radial configuration. Referring to FIG. 4A, the optical elements 154 are in the form of a rotating disk defining a planar input surface 158 that is uniform and perpendicular to the respective incident beam 114. The optical elements 154 include substantially conical wedge elements respectively defining an output surface 160 that is non-uniform, as shown in FIG. 4B. The first routing structure 122 is similarly constructed and positioned in the central region 152.

The substantially conical shape of the output surfaces 160 may impart astigmatism to the incident beams, which may be compensated by the addition of a respective corrective lens positioned along the path of the incident beams, such as cylindrical lens 162, 164 respectively positioned along the path of the incident beams 114, 118, prior to the first and second routing structure 122, 124. If the conical wedge elements in the first or second routing structures 122, 124 impart positive optical power in one direction, then the corrective lens 162, 164 corresponding to that wedge element may compensate for this added positive optical power either by adding negative optical power in the same direction or by adding positive optical power in the orthogonal direction that is 90 degrees from the original direction. For example, in FIG. 3B, element G in the second routing structure 124 adds slight positive optical power in the direction in and out of the screen (or if a hard copy, in and out of the paper). To compensate, cylindrical lens 164 may be configured as a plano-convex cylindrical lens that adds equal positive optical power but in the orthogonal direction; i.e., within the plane of the screen.

An alternative solution is to make the corrective lens 164 a plano-concave cylindrical lens where the axis of this lens is horizontal and within the plane of the screen. In this case, the cylindrical lens 164 imparts a negative optical power to the transmitted beam of a magnitude equal and opposite the optical power of element G, and where the negative optical power of cylindrical lens 164 and the power optical power of element G are in the same direction. In this case, the two optical powers essentially cancel, and the result is a beam transmitted through the second routing structure 124 that is collimated (has zero optical power) in the direction in and out of the screen. Of the two methods described above, adding negative power in the orthogonal direction has a technical advantage in that it results in a spherical wavefront with no optical power (collimated) instead of a spherical wavefront with slightly positive optical power (slightly converging). In the embodiment shown, cylindrical lenses are employed as the corrective lenses 162, 164. However, it is understood that other lenses may be employed, such as acylindrical lenses, or lenses with major cylindrical (or acylindrical) curvature in the primary direction and minor cylindrical (or acylindrical) curvature in the orthogonal direction. Furthermore, the cylindrical lens may either be planar on one lens surface, such as in FIG. 3B, or both surfaces may be cylindrically curved. Finally, a cylindrical lens with a planar surface may either have the planar surface facing the incoming beam, as in FIG. 3B, or facing away from the incoming beam.

Referring to FIG. 3A, when the first and second incident beams 114, 118 impinge upon element E of the first and second routing structures 122, 124, respectively, the output beam is directed to the first core 134. In FIG. 3B, the first and second incident beams 114, 118 impinge upon element G of the first and second routing structures 122, 124, respectively, and the output beam being directed to the third core 138. Similarly, when the first and second incident beams 114, 118 respectively impinge upon element F of the first and second routing structures 122, 124, the respective output beam is directed to the second core 136. When the first and second incident beams 114, 118 impinge upon element H (of the first and second routing structures 122, 124) respectively, the output beam 148 is directed to the fourth core 140. Thus, the first and second incident beams 114, 118 impinge upon elements E, F, G, H in sequence, and the output beam 148 is respectively directed to the first core 134, second core 136, third core 138, and fourth core 140, in sequence.

Referring to FIG. 4A-4B, each of the optical elements 154 is constructed to have a diffractive structure or refractive structure that varies across the spatial area of each element E, F, G, H in such a way that beam diffracted or refracted from each element E, F, G, H will remain unchanged, relative to the incident beam orientation, as the routing structure 124 is rotated. To do so requires that the diffractive or refractive structure changes as a function of spatial position within each of the optical elements 154 so that the changing diffracted or refracted beams are all referenced relative to the rotational axis 156 of the routing structure 124. In other words, the diffractive structure or refractive structure across an element E, F, G, H within each routing structure stays fixed along the radial direction relative to the rotational axis but changes in its orientation along the circumferential direction, such that the diffraction angle stays fixed relative to the rotational axis 156.

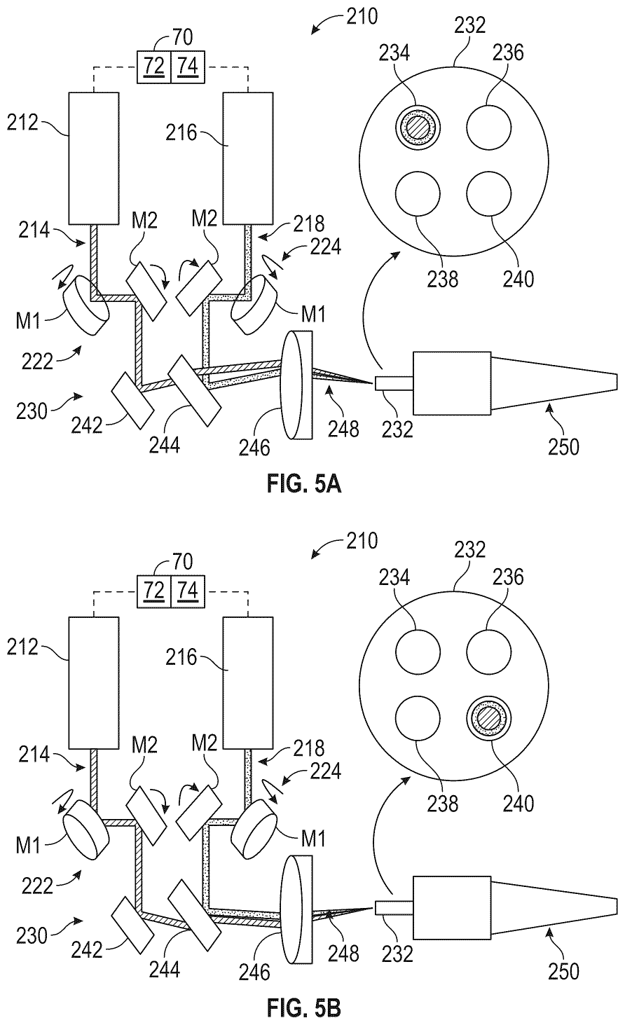

Referring now to FIGS. 5A-5B, a beam delivery system 210 in accordance with a second embodiment is shown. The beam delivery system 210 includes a first laser source 212 generating a first incident beam 214 and a second laser source 216 generating a second incident beam 218. The system 210 includes routing structures 220 respectively positioned along a path of the respective incident beam, including a first routing structure 222 in the path of the first incident beam 114 and a second routing structure 224 in the path of the second incident beam 218.

The intermediate beams exiting respectively the first and second routing structures 222, 224 are directed by an optical subsystem 230 into each core of a multi-core fiber 232 in communication with a laser probe 250, for transmission to a target site. The target site may be an anatomical location on a patient, such as the posterior region of an eye. The multi-core fiber 232 may be integrally formed with the laser probe 250. The multi-core fiber 232 may be operatively connected to the laser probe 250. In the example shown, the multi-core fiber 232 includes a first core 234, a second core 236, a third core 238, and a fourth core 240 (see FIGS. 1A, 1B). Each of the cores in the multi-core fiber 232 may act as a waveguide such that light may independently propagate through those cores. As noted above, it is understood that the number of optical elements and fiber cores may be varied based on the application at hand.

The first and second routing structures 222, 224 respectively include an array of optical elements 254 (see FIG. 5A) synchronously movable between respective positions that correspond to each core of the multi-core fiber 232. In the embodiment shown, the optical elements 254 are a pair of oscillating scanning mirrors, referred to herein as first mirror M1 and second mirror M2. In other words, the first and second routing structures 222, 224 each have a pair of oscillating scanning mirrors. The first mirror M1 and the second mirror M2 are each movable between two positions or orientations (indicated by arrows), referred to herein as P1 and P2. Thus, the four possible configurations for the first mirror M1 and the second mirror M2 are as follows: (P1, P1), (P1, P2), (P2, P1), and (P2, P2).

Referring to FIG. 5A, when the first and second incident beams 114, 118 impinge upon the first and second routing structures 222, 224 with their first and second mirrors respective in the (P1, P1) position, the output beam 248 is directed to the first core 134. Each mirror M1 (or mirror M2), as it moves from the first position P1 to the second position P2, may start out at a near-zero rate of angular tilt change, accelerate its rate of angular tilt change until the rate reaches a maximum, start decelerating its rate of angular tilt change, and finally reach zero angular tilt change as the angular tilt is at its new value corresponding to the output beam 248 pointed towards an adjacent fiber core.

As shown in FIG. 5B, when the first and second incident beams 114, 118 impinge upon the first and second routing structures 222, 224 with their first and second mirrors respective in the (P2, P2) position, the output beam 248 is directed to the fourth core 140. When the first and second incident beams 114, 118 impinge upon the first and second routing structures 222, 224 with their first and second mirrors respectively in the (P1, P2) position, the respective output beam 248 is directed to the second core 136. When the first and second incident beams 114, 118 impinge upon the first and second routing structures 222, 224 with their first and second mirrors respectively in the (P2, P1) position, the output beam 248 is directed to the third core 138.

During this process, a continuous-wave (CW) beam may remain on, and the beams trace a linear path between the starting fiber core (e.g., first core 134) and the destination fiber core (e.g., second core 136). During this linear transition, the beam may cross the absorptive, attenuative interstitial region between the adjacent fiber cores where no beam will transmit to the distal end of the multicore fiber. To avoid this, the first laser source 212 and the second laser source 216 may each be operated in pulsed mode where the time-averaged power of each laser beam equals the continuous wave power of the non-pulsed beam. Here, the transition of each pulse between zero power and maximum power and between maximum power and zero power is approximately a step function (i.e., nearly instantaneous). Thus, each incident beam 214, 218 is active or on only when the beam is residing within a core region of the multi-core fiber 32, and the laser beam power is zero during the linear transit between adjacent cores.

In the example shown, the optical subsystem 230 includes a relay mirror 242 that reflects the first incident beam 214 towards a dichroic mirror 244. The dichroic mirror 244 transmits the first incident beam 214 and reflects the second incident beam 218 towards a condensing lens 246 that refocuses an output beam 248 onto the multi-core fiber 232.

In summary, the beam delivery systems 10, 110, 210 employ a scanning optic subsystem to sequentially direct multiple incident beams respectively into multiple fiber cores, which transmit and output the beams into a condensing optical element which directs and focuses the beam or beams toward a different target location corresponding to each fiber core. The systems 10, 110, 210 do not require simultaneous transmission of split beams into multiple cores, or multiple illumination sources directed into a single core, but instead use moving optical elements 54 (which may be diffraction, reflection, or refraction elements) acting on a single incident source beam to sequentially direct the beam into multiple fiber cores, which then transmit and direct the outputs of the multiple cores to different target locations.

The technical advantages provided here with switched sequential beam inputs to multiple cores include a reduction of thermal loads in fiber optic delivery components (e.g., laser probes) by sequential multiplexing, rather than simultaneous, total power transmission of multiple beams in small gauge surgical instruments. This results in a relatively lower average power transmission and reduced laser probe tip temperature. Additionally, the systems 10, 110, 210 provide stable long-term operation and fast incident beam switching by the use of low-inertia or cyclically moving optical devices to direct the beam(s) into the multi-core fiber 32, 132, 232. This allows fast adaptation of different spot patterns in response to surgeon inputs or changing conditions.

The controller 70 may include, or otherwise have access to, information downloaded from remote sources and/or executable programs. The controller 70 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic media, a CD-ROM (Compact Disc Read Only Memory), DVD (Digital Versatile Disc), other optical media, other physical media, a RAM (Random-access memory), a PROM (Programmable Read Only Memory), an EPROM (Erasable Programmable Read Only Memory), a FLASH-EEPROM, other memory chips or cartridges, or other media from which a computer can read.

Look-up tables, databases, data repositories or other data stores described herein may include various kinds of mechanisms for storing, accessing, and retrieving various kinds of data, including a hierarchical database, a group of files in a file system, an application database in a proprietary format, a relational database management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL (Procedural Language Extensions to the Structured Query Language) language mentioned above.

The flowcharts presented herein illustrate an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based devices that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of parameters (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A beam delivery system for a probe, the system comprising:
at least one laser source configured to generate a respective incident beam, including a first laser source generating a first incident beam;
routing structures respectively positioned along a path of the respective incident beam, including a first routing structure in the path of the first incident beam, the probe being in communication with a multi-core fiber;
an optical subsystem adapted to direct a respective output beam from the routing structures into the multi-core fiber, the respective output beam being sequentially directed into each core of the multi-core fiber.

2. The system of claim 1,
wherein the at least one laser source further includes a second laser source generating a second incident beam;
wherein the routing structures further comprise a second routing structure in the path of the second incident beam;
wherein the routing structures respectively include an array of optical elements, the routing structures being adapted to be synchronously moved such that the first incident beam and the second incident beam encounter an identical member of the array at a same time.

3. The system of claim 2, wherein the optical elements include at least one of a refractive wedge, a diffractive element, and a reflective element.

4. The system of claim 2, wherein each of the routing structures are integrally formed such that the optical elements are rigidly attached to one another.

5. The system of claim 4, wherein the optical elements in the array are arranged within a plane substantially perpendicular to the respective incident beam and the routing structures are sequentially translated across each of the optical elements.

6. The system of claim 5, wherein each of the optical elements is configured to have a diffractive structure or a refractive structure that is fixed across a spatial area of the optical elements.

7. The system of claim 4, wherein:
the routing structures respectively include a rotating disk defining a planar input surface perpendicular to the respective incident beam, the optical elements being arranged in a radial configuration; and
the routing structures are sequentially rotated across each of the optical elements along a rotational axis perpendicular to the plane of the rotating disk.

8. The system of claim 7, wherein the optical elements include substantially conical wedge elements respectively defining an output surface.

9. The system of claim 7, wherein the optical elements are configured to have a refractive structure that varies along a circumferential direction relative to the rotational axis, the refractive structure being fixed along a radial direction relative to the rotational axis.

10. The system of claim 7, further comprising:
a respective corrective lens positioned along the path of the respective incident beam, prior to the respective incident beam encountering the routing structures.

11. The system of claim 2, wherein the optical elements include oscillating scanning mirrors synchronously movable between respective positions corresponding to each core of the multi-core fiber.

12. The system of claim 1, further comprising:
a controller configured to pulse the first and second laser sources such that each laser beam generated by the first and second laser sources is only active when the laser beam is residing within a respective core region of the multi-core fiber; and
wherein the controller has at least one processor and at least one non-transitory, tangible memory on which instructions are recorded; and
wherein a time-averaged power of the laser beam equals a continuous wave power of a non-pulsed beam.

13. A beam delivery system for a probe, the system comprising:
a plurality of laser sources configured to generate a respective incident beam, including a first laser source generating a first incident beam and a second laser source generating a second incident beam;
routing structures respectively positioned along a path of the respective incident beam, including a first routing structure in the path of the first incident beam and a second routing structure in the path of the second incident beam, the probe being in communication with a multi-core fiber;
an optical subsystem adapted to sequentially direct a respective output beam from the routing structures respectively into each core of the multi-core fiber; and
wherein the first routing structure and the second routing structure respectively include an array of optical elements synchronously movable between respective positions corresponding to each core of the multi-core fiber.

14. The system of claim 13, wherein the optical elements are oscillating scanning mirrors.

15. The system of claim 14, further comprising:
a controller configured to pulse the plurality of laser sources such that each laser beam generated by the plurality of laser sources is only active when the laser beam is residing within a respective core region of the multi-core fiber; and
wherein the controller has at least one processor and at least one non-transitory, tangible memory on which instructions are recorded.
